(19) United States
(12) Patent Application Publication
(10) Pub. No.: US 2020/0164115 A1
(43) Pub. Date: May 28, 2020

(54) ELECTRODE FOR HIGH-FREQUENCY MEDICAL DEVICE AND HIGH-FREQUENCY MEDICAL DEVICE

(71) Applicant: OLYMPUS CORPORATION, Tokyo (JP)

(72) Inventors: Yu MURANO, Hirosaki-shi (JP); Yoshiyuki OGAWA, Tokyo (JP); Hiroaki KASAI, Tokyo (JP); Takuya FUJIHARA, Tokyo (JP)

(73) Assignee: OLYMPUS CORPORATION, Tokyo (JP)

(21) Appl. No.: 16/777,917

(22) Filed: Jan. 31, 2020

Related U.S. Application Data
(63) Continuation of application No. PCT/JP2018/038659, filed on Oct. 17, 2018.

(30) Foreign Application Priority Data
Oct. 25, 2017 (JP) 2017-206552

(51) Int. Cl. A61L 31/12; A61B 18/14; A61L 31/10; A61L 31/02; A61L 31/14

(52) U.S. Cl. CPC A61L 31/128; A61B 18/14; A61L 31/10; A61B 2018/00601; A61L 31/14; A61B 2018/1412; A61L 31/022

(57) ABSTRACT

An electrode for high-frequency medical device, has a substrate; an intermediate layer laminated on the substrate, wherein the intermediate layer has a top layer formed on the outermost part of the intermediate layer and the top layer is formed from a metal layer having a higher thermal conductivity than that of the substrate; and a coating layer laminated on the intermediate layer, wherein the coating layer is configured such that a plurality of metal particles having a thermal conductivity equal to or larger than 250 W/(m·K) are distributed in a nonmetal material.

This application is a continuation application of a PCT International Application No. PCT/JP2018/038659, filed on Oct. 17, 2018, whose priority is claimed on a Japanese Patent Application No. 2017-206552, filed on Oct. 25, 2017. The contents of both the PCT International Application and the Japanese patent application are incorporated herein by reference.

### BACKGROUND

### Field of the Invention

This invention relates to an electrode for a high-frequency medical device and a high-frequency medical device having the same.

### Description of Related Art

A device configured to apply high-frequency voltage to biological tissues as a high-frequency medical device is known.

For example, a high-frequency treatment tool used as the high-frequency medical device is configured to dissect the biological tissues, coagulate the biological tissues, and perform cautery with respect to the tissues by applying the high-frequency voltage to the biological tissues.

For example, in Japanese Unexamined Patent Application, First Publication No. 2009-445, it is disclosed to form a coating layer formed from polytetrafluoroethylene (PTFE) having the nickel or a coating layer formed from PTFE having the aurum on a surface of a main body of an electrode so as to prevent the thrombus from adhering thereto.

### SUMMARY

According to a first aspect of the present invention, an electrode for high-frequency medical device has a substrate; an intermediate layer laminated on the substrate, wherein the intermediate layer has a top layer formed on the outermost part of the intermediate layer and the top layer is formed from a metal layer having a higher thermal conductivity than that of the substrate; and a coating layer laminated on the intermediate layer, wherein the coating layer is configured such that a plurality of metal particles having a thermal conductivity equal to or larger than 250 W/(m·K) are distributed in a nonmetal material.

According to a second aspect of the present invention, in the electrode for high-frequency medical device according to the first aspect, the plurality of metal particles may be categorized into at least two groups including a first metal-particle group having a first median diameter and a second metal-particle group having a second median diameter larger than the first median diameter.

According to a third aspect of the present invention, in the electrode for high-frequency medical device according to the second aspect, the first median diameter may be equal to or larger than 0.01 micrometres and equal to or less than 0.5 micrometres, and the second median diameter may be equal to or larger than 5 micrometres and equal to or less than 20 micrometres.

According to a fourth aspect of the present invention, in the electrode for high-frequency medical device according to the second or the third aspect, wherein according to a volume-based cumulative distribution, a particle diameter corresponding to 5% of an accumulation from a small-diameter side toward a large-diameter side is represented as D5 and the particle diameter corresponding to 95% of the accumulation from the small-diameter side toward the large-diameter side is represented as D95, a value of D95 may be equal to or less than 1.0 micrometre in the first metal-particle group, and a value of D5 may be equal to or larger than 3 micrometres, and a value of D95 is equal to or less than 35 micrometres in the second metal-particle group.

According to a fifth aspect of the present invention, in the electrode for high-frequency medical device according to anyone of the second to the fourth aspect, the first metal-particle group and the second metal-particle group may be included in the coating layer by a content percentage equal to or more than 10 vol % and equal to or less than 80 vol %.

According to a sixth aspect of the present invention, in the electrode for high-frequency medical device according to any one of the second to the fifth aspect, a volume ratio of the first metal-particle group with respect to the second metal-particle group may be equal to or more than 0.2 and equal to or less than 4.5.

According to a seventh aspect of the present invention, in the electrode for high-frequency medical device according to any one of the second to the sixth aspect, the nonmetal material of the coating layer may include at least one of a group consisting from fluororesin, silicone resin, polyetheretherketone resin, and ceramic.

According to an eighth aspect of the present invention, in the electrode for high-frequency medical device according to any one of the second to the seventh aspect, a thickness of the intermediate layer may be equal to or more than 5 micrometres and equal to or less than 100 micrometres.

According to a ninth aspect of the present invention, in the electrode for high-frequency medical device according to any one of the second to the eighth aspect, the electrode may include at least one of a group consisting from a metal material having aluminum, a metal material having titanium, and stainless steel.

According to a tenth aspect of the present invention, a high-frequency medical device has the electrode according to any one of the second to the ninth aspect.

### BRIEF DESCRIPTION OF DRAWING

FIG. 1 is a schematic view showing a configuration of an example of a high-frequency medical device according to an embodiment of the present invention.

FIG. 2 is a sectional view along the line A-A in FIG. 1.

FIG. 3 is a schematic sectional view showing an electrode of the high-frequency medical device according to the embodiment.

FIG. 4 is a schematic sectional view showing an electrode of the high-frequency medical device according to a modification example.

### DETAILED DESCRIPTION OF EMBODIMENTS

An electrode for a high-frequency medical device and the high-frequency medical device according to an embodiment of the present invention will be described by referring to the enclosed figures.

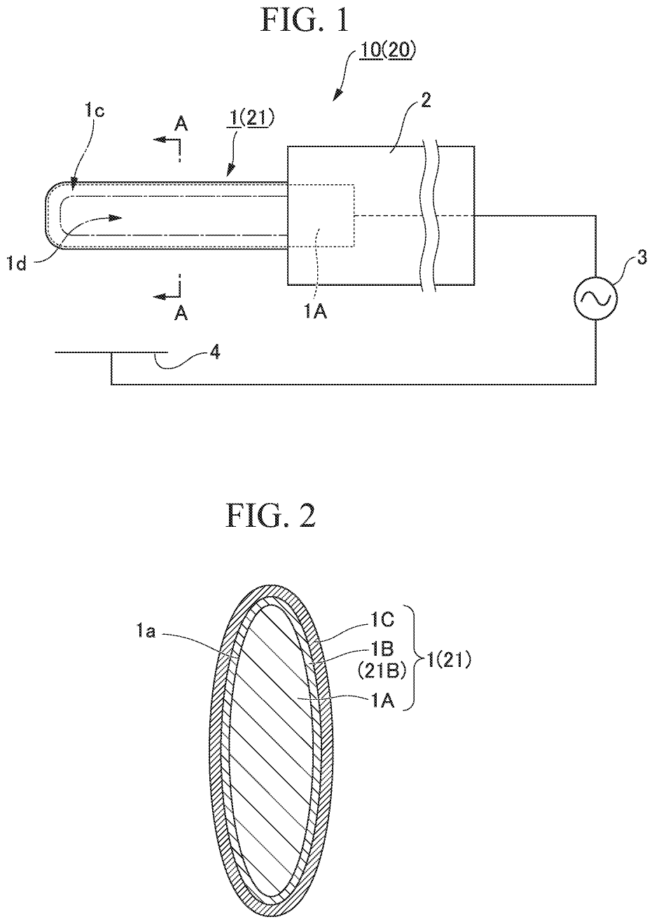

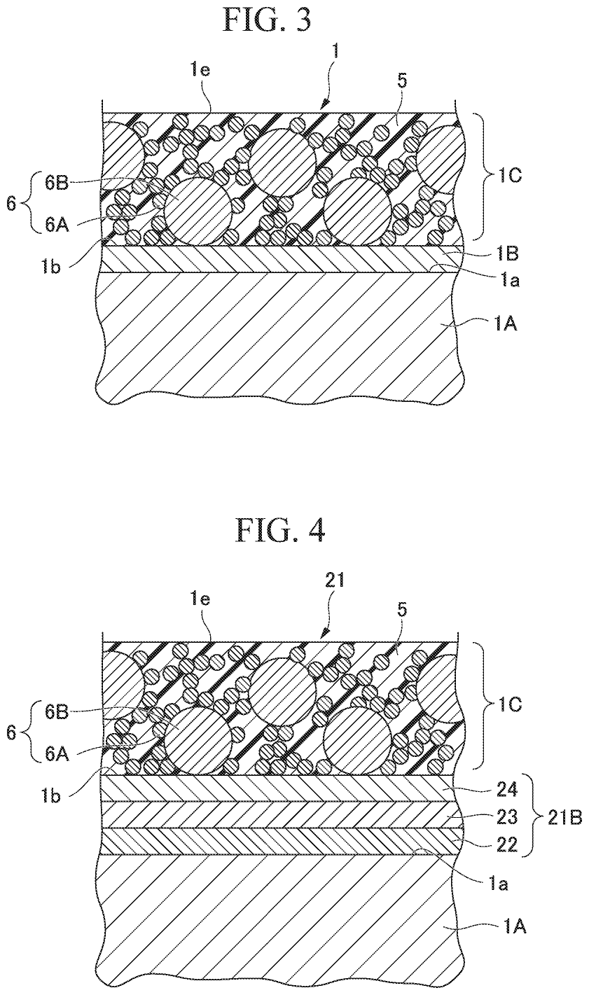

FIG. 1 is a schematic view showing a configuration of an example of a high-frequency medical device according to an embodiment of the present invention. FIG. 2 is a sectional view along the line A-A in FIG. 1. FIG. 3 is a schematic sectional view showing an electrode of the high-frequency medical device according to the embodiment.

A high-frequency knife 10 according to the present embodiment as shown in FIG. 1, is an example of the high-frequency medical device according to the embodiment. The high-frequency knife 10 is the medical treatment tool configured to dissect and remove the biological tissues, coagulate the biological tissues (hemostasis), and perform cautery with respect to the biological tissues by applying the high-frequency voltage to the biological tissues.

The high-frequency knife 10 has a rod-shaped grasping portion 2 for a surgeon to grasp by hand and an electrode portion 1 (an electrode for the high-frequency medical device) protruded from a distal end of the grasping portion 2.

The electrode portion 1 is configured to come in contact with the biological tissues as the treatment target to apply the high-frequency voltage thereto.

The electrode portion 1 has a blade portion 1c with an outer edge portion suitable for dissecting the biological tissues. A lateral surface surrounded by the blade portion 1c in the electrode portion 1 forms an abdomen portion 1d suitable for coagulating the tissues and similar surgical processes. The abdomen portion 1d is formed from a flat surface or a gently curved surface close to a flat surface.

However, the shape shown in FIG. 1 and FIG. 2 is only an example of the electrode portion 1. For example, the electrode portion 1 may be formed in a round rod shape, a square bar shape, a disc shape, a hook shape, and the like.

As shown in FIG. 2, the electrode portion 1 having an electrode main body 1A (substrate), an intermediate layer 1B, and a coating layer 1C.

As shown in FIG. 1, an outer shape of the electrode main body 1A is formed in a rectangular plate shape that has an arc-shaped portion in a distal corner portion in the protrusion direction of the electrode portion 1.

As shown in FIG. 2, in a cross section of the electrode main body 1A that is orthogonal to the protrusion direction (a direction from back side toward front side in a depth direction of the paper as shown in figures), the electrode main body 1A is formed in a flat shape such that a thickness of the electrode main body 1A becomes smaller toward the outer edge thereof. Although it is not shown in figures, a cross section of the outer edge portion of the electrode main body 1A at the distal end side (left side of the electrode portion 1 in FIG. 1) of the protrusion direction is similar that the thickness thereof becomes smaller toward the outer edge.

In the example shown in FIG. 2, the outer edge portion of the electrode main body 1A is rounded in the cross section orthogonal to the protrusion direction. A curvature radius of the rounded shape of the outer edge portion is suitably determined due to the usage of the high-frequency knife 10. In FIG. 2, an example of setting the curvature radius of the rounded shape of the outer edge portion to be about a quarter of the thickness of the electrode main body 1A is shown. However, the curvature radius of the rounded shape of the outer edge portion is not limited thereto and may be set smaller or larger. The curvature radius of the rounded shape may be enough small to form a sharp edge.

A suitable metal material having both conductivity and good workability is adopted as a material of the electrode main body. Unless otherwise mentioned in this specification, the “metal material” refers to a metal or an alloy. In this specification, the metal material described by the chemical element refers to an elementary metal of a high purity unless the metal material is described as the alloy.

For example, the electrode main body 1A may be formed from a metal material having a thermal conductivity lower than 250 W/(m·K). Unless otherwise mentioned in this specification, the value of the thermal conductivity is described by the value measured at 20 degree Celsius.

A metal material having the stainless steel and the aluminum, a metal material having the titanium, and the like are considered to be suitable metal materials for the electrode main body 1A. For example, the metal material having the stainless steel and the aluminum, and the metal material having the titanium have good workability such that it is easy to manufacture the electrode main body 1A having a complex shape.

For example, the thermal conductivities of the stainless steel such as SUS303, SUS304, and the like according to the Japanese Industrial Standards, the aluminum, and the titanium are 17-21 W/(m·K), 204 W/(m·K), and 17 W/(m·K), respectively.

As shown in FIG. 1, the electrode main body 1A is electrically connected to a high-frequency power source 3 via an electric wiring, wherein the electric wiring is connected to a proximal end portion held by the grasping portion 2. An indifferent plate 4 attached to the treatment target is electrically connected to the high-frequency power source 3.

As shown in FIG. 2 and FIG. 3, the intermediate layer 1B is a thin film laminated on an electrode main body surface 1a so as to at least coat the whole part of the electrode main body 1A protrude from the grasping portion 2.

The intermediate layer 1B may have a single layer structure or a multi-layer structure. The intermediate layer 1B may have an inclined layer formed with a composition which contents are varying in a thickness direction thereof. In the example shown in FIG. 3, the intermediate layer 1B is formed in the single layer structure.

The thickness of the intermediate layer 1B is more preferable to be equal to or more than 5 micrometres and equal to or less than 100 micrometres.

When the thickness of the intermediate layer 1B is less than 5 micrometres, thermal accumulation is easy to occur in the coating layer 1C such that the temperature of the coating layer 1C may be extremely high.

When the thickness of the intermediate layer 1B is more than 100 micrometres, cracks by the elastic deformation due to the stress during the dissection may occur in the intermediate layer 1B so as to lead to a peeling of the surface layer of the electrode portion 1.

The intermediate layer 1B is configured to have a metal layer formed from the metal material having a higher thermal conductivity than that of the electrode main body 1A at least at a top layer thereof. In a case in which the intermediate layer 1B has the multi-layer structure, it is preferable that each layer of the intermediate layer 1B is formed by the metal material. It is more preferable that the metal material used in the intermediate layer 1B has a smaller electrical conductivity than that of the electrode main body 1A.

It is more preferable that the intermediate layer 1B is formed from a material having good adhesive characteristic with the electrode main body 1A (coating layer 1C) at a bonding surface with respect to the electrode main body 1A (coating layer 1C).

For example, a metal material included in metal particles 6 of the coating layer 1 described below may be used to form the metal layer of the top layer of the intermediate layer 1B. In this case, suitable adhesive characteristic can be achieved since the same kind of metal is in close contact with each other.

The intermediate layer 1B shown in FIG. 3 has a single layer structure such that the whole intermediate layer 1B is formed by a metal layer having a larger thermal conductivity than that of the electrode main body 1A.

The thermal conductivity of the intermediate layer 1B is preferable to be equal to or larger than 200 W/(m·K), and is further preferable to be equal to or larger than 250 W/(m·K).

When the thermal conductivity is equal to or larger than 200 W/(m·K), for example, in the case in which the electrode main body 1A is formed by the metal material having the stainless steel and the titanium, the thermoconductive of the intermediate layer 1B is significantly improved comparing to that of the electrode main body 1A.

When the thermal conductivity is equal to or larger than 250 W/(m·K), for example, in the case in which the electrode main body 1A is formed by the metal material having the aluminum, the thermoconductive of the intermediate layer 1B is increased comparing to that of the electrode main body 1A.

Examples of the metal materials which are suitably used in the metal layer of the intermediate layer 1B are given as the argentum, the aurum, the cuprum, the aluminum, and alloy having such metal materials. The thermal conductivities of the argentum, the aurum, and the cuprum are 418 W/(m·K), 295 W/(m·K), and 386 W/(m·K), respectively.

The intermediate layer 1B is coated by a coating layer 1C described below such that the intermediate layer 1B does not come in contact with the biological tissues. Accordingly, the material of the intermediate layer 1B is not necessary to be the material superior in biocompatibility.

As shown in FIG. 3, the coating layer 1C is laminated on a top surface 1b of the intermediate layer 1B, and the coating layer 1C is a layered portion by distributing metal particles 6 having a thermal conductivity equal to or large than 250 W/(m·K) in a base material 5 (nonmetallic material).

The coating layer 1C is configured to form an outermost surface of the electrode portion 1 at least in a region in contact with the biological tissues (see FIG. 2). According to the present embodiment, the coating layer 1C at least covers the intermediate layer 1B of the electrode main body 1A protruding from the grasping portion 2.

The base material 5 is configured to have fine adhesion with the top surface 1b of the intermediate layer 1B, and the base material 5 is configured by the nonmetallic material which is difficult for the biological tissues to be adhered thereto. For example, the base material 5 is preferable to include at least one of a group consisting from the fluororesin, the silicone resin, the polyetheretherketone resin, and the ceramic.

In the present embodiment, the metal particles are formed from a first metal-particle group and a second metal-particle group. The first metal-particle group is a group of particles having a first median diameter. The second metal-particle group is a group of particles having a second median diameter larger than the first median diameter.

Here, the recitation “median diameter” represents a particle diameter corresponding to 50% (D50) of the accumulation from the small-diameter side toward the large-diameter side in the volume-based cumulative distribution.

The first metal-particle group and the second metal-particle group have different median diameters such that the metal particles 6 as a whole have a bimodal particle diameter distribution.

For example, the first median diameter is more preferable to be equal to or more than 0.01 micrometres and equal to or less than 0.5 micrometres. The second median diameter is more preferable to be equal to or more than 5 micrometres and equal to or less than 20 micrometres. It is further more preferable that the first median diameter is further preferable to be equal to or more than 0.01 micrometres and equal to or less than 0.5 micrometres, and the second median diameter is further preferable to be equal to or more than 5 micrometres and equal to or less than 20 micrometres.

It is further more preferable that the particle diameter distribution of the first metal-particle group and the particle diameter distribution of the second metal-particle group have less overlapped region, or do not overlap with each other. For example, according to the volume-based cumulative distribution, when the particle diameter corresponding to 5% of the accumulation from the small-diameter side toward the large-diameter side is represented as D5 and the particle diameter corresponding to 95% of the accumulation from the small-diameter side toward the large-diameter side is represented as D95, it is preferable that the D95 in the first metal-particle group is equal to or smaller than 1.0 micrometre, while the D5 in the second metal-particle group is equal to or larger than 3 micrometres and equal to or smaller than 35 micrometres.

In the present embodiment, as shown in FIG. 3, the metal particles 6 are formed from a plurality of first particles 6A belonging to the first metal-particle group and a plurality of second particles 6B belonging to the second metal-particle group.

The plurality of first particles 6A and the plurality of second particles 6B may be formed from different materials or the same material.

In the case in which the plurality of first particles 6A and the plurality of second particles 6B are formed from different materials, the plurality of first particles 6A and the plurality of second particles 6B can be distinguished by the physical characteristic thereof. Accordingly, for example, even in a state in which the plurality of first particles 6A and the plurality of second particles 6B are mixed in the coating layer 1C, it is possible to distinguish the plurality of first particles 6A and the plurality of second particles 6B with each other and measure the particle diameter distribution of each particle group. The particle diameter distribution may be statistically estimated by sampling.

In the case in which the plurality of first particles 6A and the plurality of second particles 6B are formed from the same material, the plurality of first particles 6A and the plurality of second particles 6B cannot be distinguished from each other except for the difference of the particle diameter. In this case, the particle diameter distribution of the whole metal particles 6 is measured first.

In a case in which the particle diameter distribution is dissociated in two or more groups since a discontinuous portion is existed in the particle diameter distribution, each particle diameter distribution of the first metal-particle group and the second metal-particle group is specified by suitably dividing the group of particles into two groups with the discontinuous portion as a boundary.

In a case in which the particle diameter distribution does not have the discontinuous portion and the metal particles 6 are divided into the first metal-particle group and the second metal-particle group, the particle diameter distribution has the bimodal characteristic.

In this case, for example, it is considerable to divide the particle diameter distribution by performing the curve fitting. However, in a case in which the particle diameter distributions of the first metal-particle group and the second metal-particle group have less overlapped region, the particle group may be divided into two groups with a particle diameter having the minimum distribution as the boundary between two dominant peaks.

According to such a configuration, in the case in which the plurality of first particles 6A and the plurality of second particles 6B are formed from the same material and mixed together, the first median diameter and the second median diameter, and representative values of the particle distribution of the first metal-particle group and the second metal-particle group are measured.

In the coating layer 1C, it is more preferable to include the metal particles 6 equal to or more than 10 vol % and equal to or less than 80 vol %. Here, the recitation “vol %” refers to the volume ratio.

When a content percentage of the metal particles 6 in the coating layer 1C is less than 10 vol %, contact positions of the metal particles 6 becomes less such that the heat dissipation of the coating layer 1C is degraded.

When the content percentage of the metal particles 6 in the coating layer 1C is more than 80 vol %, a viscosity of the paint used for forming the coating layer 1C is increased such that it is difficult to form the coating layer 1C by painting.

In the coating layer 1C, the volume ratio of the first metal-particle group with respect to the second metal-particle group is more preferable to be set to be equal to or more than 0.2 and equal to or less than 4.5. For example, when the volume content ratio of the first metal-particle group is represented as A and the volume content ratio of the second metal-particle group is represented as B, a proportion of A to B coincides with a volume proportion of the first metal-particle group to the second metal-particle group.

When the volume proportion of the first metal-particle group to the second metal-particle group is less than 0.2, since the volume content of the first metal-particle group is not enough with respect to the volume content of the second metal-particle group, an amount of the plurality of first particles 6A filled into the gaps among the plurality of second particles 6B and the gaps between the plurality of second particles 6B and the top surface 1b of the intermediate layer 1B is not enough. In this case, a contact amount of the metal particles 6 in the coating layer C and a contact amount of the metal particles 6 and the top surface 1b of the intermediate layer 1B are not enough such that the thermal conductivity of the coating layer 1C is degraded.

When volume proportion of the first metal-particle group to the second metal-particle group is more than 4.5, since the volume content of the first metal-particle group is too much with respect to the volume content of the second metal-particle group, the viscosity of the paint used for forming the coating layer 1C is increased. Thus, it is difficult to form the coating layer 1C by painting.

The materials of the plurality of first particle 6A and the plurality of second particle 6B only has to have a thermal conductivity equal to or more than 250 W/(m·K), and the materials thereof are not particularly limited. It is possible that the plurality of first particles 6A and the plurality of second particles 6B form a part of an outer circumferential surface 1e of the coating layer 1C exposed from the base material 5. Accordingly, it is more preferable to use the metal material having biocompatibility and being difficult for the biological tissues to adhere to form the plurality of first particles 6A and the plurality of second particles 6B.

Examples of the suitable material for forming the plurality of first particles 6A and the plurality of second particles 6B are given as the metal material including the argentum, the aurum, and the cuprum.

The electrode portion 1 described above may be manufactured by the following method, for example.

For example, a suitable metal material is processed so as to manufacture the electrode main body 1A. The manufacture method of the electrode main body 1A can be press processing, incision processing, mold processing and the like.

Subsequently, the intermediate layer 1B is formed on the electrode main body surface 1a of the electrode main body 1A.

The method of forming the intermediate layer 1B can be plating, Physical Vapor Deposition (PVD), Chemical Vapor Deposition (CVD), and the like.

Subsequently, the coating layer 1C is formed on the top surface 1b of the intermediate layer 1B.

The coating layer 1C is formed by the painting process for example. In this case, firstly, the plurality of first particles 6A and the plurality of second particles 6B are mixed in plastic paint or ceramic paint having the ingredient of the base material 5. Accordingly, the paint used for forming the coating layer 1C is formed.

Subsequently, the paint is painted on the top surface 1b of the intermediate layer 1B by suitable painting means.

The painting means is not particularly limited. Examples of the painting means can be given as spraying, dip coating, spin coating, screen printing, ink-jet technology, flexographic printing, gravure printing, pad printing, hot stamp, and the like. According to the spraying and the dip coating, it is easy to perform the painting process even if the shape of the target for painting is complex, thus the spraying and the dip coating are particularly suitable as the painting means to form the coating layer 1C of the high-frequency medical device.

For example, the layer of the paint formed on the intermediate layer 1B can be dried by heat. Thus, the coating layer 1C is formed.

As described above, the electrode portion 1 is manufactured.

Next, effects of the high-frequency knife 10 and the electrode portion 1 having such configurations will be described.

The surgical process using the high-frequency knife 10 is performed, for example, in a state in which the indifferent plate 4 is fitted to the patient and the high-frequency voltage is applied to the electrode portion 1 by the high-frequency power source 3. In the state in which the high-frequency voltage is applied to the electrode portion 1, the surgeon makes the blade portion 1c or the abdomen portion 1d of the electrode portion 1 to come in contact with the treatment target such as the treatment target portion of the patient.

When the high-frequency voltage is applied between the electrode portion 1 and the indifferent plate 4, high-frequency current is generated between the electrode portion 1 and the biological tissues via the coating layer 1C. Joule heat is generated due to the high-frequency current flowing through the biological tissues. Accordingly, the moisture of the biological tissues as the treatment target rapidly evaporates such that the biological tissues are cut due to the press force from the blade portion 1c. Thus, it is possible to dissect and remove the biological tissues by moving the electrode portion 1 with respect to the biological tissues.

When the high-frequency current flows in the state in which the abdomen portion 1d is pressed to be in contact with the treatment target, the moisture of the biological tissues as the treatment target rapidly evaporates such that the biological tissues in the vicinity of the abdomen portion 1d is coagulated. Accordingly, it is possible to perform the hemostasis and the cautery with respect to the biological tissues by pressing the abdomen portion 1d to the treatment target.

When the necessary treatment is finished, the surgeon separates the electrode portion 1 from the treatment target. At this time, since it is difficult for the biological tissues to adhere to the outer surface 1e of the coating layer 1C in contact with the biological tissues due to the base material 5, it is easy to peel the biological tissues.

Due to terms of use of the high-frequency knife 10, the base material 5 is exposed in the high-temperature environment due to the heat generated by the high-frequency current. For example, by applying the high-frequency voltage, when the electric discharge is caused on the surface of the electrode portion 1, discharge energy is concentrated in the minute area of the base material 5 such that it is possible for the temperature to exceed the heat-resistant temperature of the base material 5 locally. When the base material 5 is exposed to the high-temperature environment, denaturation of the base material 5 occurs such that the adhesion preventing characteristic with respect to the biological tissues deteriorates.

In the present embodiment, when the coating layer 1C is heated, the heat dissipation occurs through the metal particles 6 in contact with each other. The metal particles 6 have the thermal conductivity equal to or more than 250 W/(m-K) to have favorable thermal conductive characteristic. Thus, the metal particles 6 in contact with each other form an efficient heat dissipation path.

The metal particles 6 are distributed in the base material 5 such that a plurality of heat dissipation paths are formed to cross the coating layer 1C in the thickness direction in accordance with the content amount of the metal particles 6. Accordingly, the heat inside the coating layer 1C is thermally transmitted to the top surface 1b of the intermediate layer 1B via the metal particles 6 on the bottom of the coating layer 1C.

The metal layer having a higher thermal conductivity than the electrode main body 1A is formed in the top surface 1b of the intermediate layer 1B such that the heat transmitted to the top surface 1b at least thermally transmits to the metal layer and spreads in the metal layer. Particularly in the present embodiment, the whole intermediate layer 1B is formed as the metal layer. Furthermore, the intermediate layer 1B is formed covering the whole surface of the electrode main body 1A.

The heat transmitted from the metal particles 6 rapidly transmits and spreads in the surface direction of the intermediate layer 1B such that the heat transmitted from the metal particles is dissipated to a low-temperature region from the high-temperature treatment portion.

As a result, even if the electrode main body 1A is formed from the material with a low thermal conductivity, for example, the metal material such as the stainless steel, the titanium, and the like, high heat dissipation characteristic can be achieved due to the intermediate layer 1B. Thus, the temperature rise of the base material 5 in the coating layer 1C is suppressed.

In the electrode portion 1, the temperature rise of the base material 5 is suppressed and the denaturation of the base material 5 due to the temperature rise is suppressed. Accordingly, the adhesion preventing characteristic of the base material 5 with respect to the biological tissues is maintained for a long period.

Particularly in the present embodiment, there is a case in which the metal particles 6 are formed from the first metal-particle group having the first median diameter and the second metal-particle group having the second median diameter. In this case, since the particle diameter of each of the plurality of first particles 6A is small, for example, the plurality of first particles 6A enter the gaps generated due to the contact among the plurality of second particles 6B so as to come in contact with the plurality of second particles 6B. As a result, the contact path between the adjacent second particles 6B increases since the plurality of first particles 6A belonging to the first metal-particle group come in contact with the circumference of the plurality of second particles 6B belonging the second metal-particle group.

The smaller the particle diameter of the first particle 6A is, the more the contact points with the plurality of second particles 6B increase, such that more heat dissipation paths are formed. However, it is possible that the viscosity of the paint used for forming the coating layer 1C becomes too large if the volume content ratio of the plurality of first particles 6A is too large.

In order to achieve both of the adhesion preventing characteristic with respect to the biological tissues and the manufacturability, for example, it is more preferable to set each median diameter, particle diameter distribution, volume content ratio and the like of the first metal-particle group and the second metal-particle group to a more preferable range than the above-described ranges.

As described above, the high-frequency knife 10 and the electrode portion 1 according to the present embodiment can maintain the adhesion preventing characteristic with respect to the biological tissues for a long period. Accordingly, the endurance period of the high-frequency knife 10 and the electrode portion 1 is improved.

### Modification Example

An electrode for high-frequency medical device and a high-frequency medical device according to a modification example of the present embodiment will be described.

FIG. 4 is a schematic sectional view showing an electrode of the high-frequency medical device according to the modification example of the present embodiment.

As shown in FIG. 4, a high-frequency knife (high-frequency medical device) 20 according to the present modification example has an electrode portion (electrode for high-frequency medical device) 21 instead of the electrode portion 1 according to the above-described embodiment. As shown in FIG. 4, the electrode portion 21 according to the present modification example has an intermediate layer 21B instead of the intermediate layer 1B of the electrode portion 1 according to the above-described embodiment.

Hereinafter, configurations will be described with a focus on the differences with the above-described embodiments.

As shown in FIG. 4, the intermediate layer 21B has a configuration of laminating a first metal layer 22, a second metal layer 23, and a second metal layer (top layer, metal layer) 24 in this sequence from the electrode main body surface 1a toward the top surface 1b of the electrode main body 1A. Accordingly, the intermediate layer 21B is an example of having a multi-layer structure.

Except for that the third metal layer 24 is formed from a metal material having a higher thermal conductivity than that of the electrode main body 1A, the materials and the layer thicknesses of the first metal layer 22, the second metal layer 23, and the third metal layer 24 are not particularly limited.

Since the intermediate layer 21B has the multi-layer structure, it is possible to change the materials of the first metal layer 22 in contact with the electrode main body 1A and the third metal layer 24 in contact with the coating layer 1C. Accordingly, even if there is no material which is able to come in close contact with both of the electrode main body 1A and the coating layer 1C and has a suitable thermal conductivity, it is possible to achieve the favorable adhesion between the intermediate layer 21B and each of the electrode main body 1A or the coating layer 1C.

For example, if the second metal layer 23 is formed from the alloy having the metal composition of the first metal layer 22 and the metal composition of the third metal layer 24, both the adhesion characteristic between the electrode main body 1A and the first metal layer 22 and the adhesion characteristic between the coating layer 1C and the third metal layer 24 become favorable.

The materials of the first metal layer 22, the second metal layer 23, and the third metal layer 24 may be selected from combinations of materials in which the electrolytic corrosion is difficult to occur between the contact partners. In this case, since the electrolytic corrosion is suppressed, the durability of the electrode portion 1 is further improved.

The materials of the first metal layer 22, the second metal layer 23, and the third metal layer 24 may be selected from materials whose difference of the thermal expansion coefficients on each interface and on the interface with the electrode main body 1A is small. In this case, the load due to the thermal stress becomes less such that the durability of the electrode portion 1 is further improved.

The high-frequency knife 20 according to the present modification example is different from the above-described embodiment only in that the intermediate layer 21B has a multi-layer structure, accordingly, similar to the above-described embodiment, the adhesion preventing characteristic with respect to the biological tissues can be maintained for a long period.

In the description of the embodiment and the modification example, the example of the high-frequency knife configured as the high-frequency medical device having the electrode for high-frequency medical device is described, however, the high-frequency medical device is not particularly limited to the high-frequency knife. Other examples of the high-frequency medical devices to which the electrode for high-frequency device of the present invention can be suitably used can be given as the treatment tools such as the electrocautery, the bipolar tweezers, the probe, the snare and the like.

In the description of the embodiment and the modification example, the example of the case in which the metal particles 6 are formed from the plurality of first particles 6A and the plurality of second particles 6B are described; however, the particle distribution of the metal particles 6 may be a unimodal distribution if the necessary heat dissipation path is formed by the contact of the metal particles 6 in the coating layer 1C.

### EXAMPLES

Next, Examples 1-17 of the electrode for high-frequency medical device according to the above-described embodiment will be described together with Comparison Examples 1-4. The configurations according to each example and each comparison example are shown in Table 1 and Table 2 as follows.

TABLE 1

| | | INTERMEDIATE LAYER | |
|---|---|---|---|
| | | | LAYER |
| | SUBSTRATE | | THICKNESS |
| | MATERIAL | MATERIAL | (μm) |
| EXAMPLE 1 | SUS304 | Ag | 7 |
| EXAMPLE 2 | SUS304 | Ag | 7 |
| EXAMPLE 3 | SUS304 | Ag | 7 |
| EXAMPLE 4 | SUS304 | Ag | 7 |
| EXAMPLE 5 | SUS304 | Ag | 7 |
| EXAMPLE 6 | SUS304 | Ag | 7 |
| EXAMPLE 7 | SUS304 | Ag | 7 |
| EXAMPLE 8 | SUS304 | Ag | 5 |
| EXAMPLE 9 | SUS304 | Ag | 30 |
| EXAMPLE 10 | SUS304 | Ag | 100 |
| EXAMPLE 11 | SUS304 | Ag | 7 |
| EXAMPLE 12 | SUS304 | Ag | 7 |
| EXAMPLE 13 | SUS304 | Ag | 7 |
| EXAMPLE 14 | SUS304 | Al | 7 |
| EXAMPLE 15 | SUS304 | Au | 7 |
| EXAMPLE 16 | SUS304 | Cu | 7 |
| EXAMPLE 17 | SUS304 | Ag | 7 |
| COMPARISON | SUS304 | Ag | 7 |
| EXAMPLE 1 | | | |
| COMPARISON | SUS304 | Ag | 7 |
| EXAMPLE 2 | | | |
| COMPARISON | SUS304 | Ag | 7 |
| EXAMPLE 3 | | | |
| COMPARISON | SUS304 | — | — |
| EXAMPLE 1 | | | |

TABLE 2
COAT LAYER

| | | BASE | | FIRST METAL | | | | | SECOND METAL | | | | | |
|---|---|---|---|---|---|---|---|---|---|---|---|---|---|---|
| | | MATERIAL | | PARTICLE GROUP | | | | | PARTICLE GROUP | | | | | |
| | | | CONTENT | | | | | CONTENT | | | | | CONTENT | |
| | LAYER | | PER- | | | | | PER- | | | | | PER- | VOL- |
| | THICK- | MA- | CENT- | MA- | | | | CENT- | MA- | | | | CENT- | UME |
| | NESS | TERI- | AGE | TERI- | D50 | D5 | D95 | AGE | TERI- | D50 | D5 | D95 | AGE | RATIO |
| | (μm) | AL | (vol %) | AL | (μm) | (μm) | (μm) | (vol %) | AL | (μm) | (μm) | (μm) | (vol %) | A/B |
| EXAMPLE 1 | 32 | Sil | 40 | Ag | 0.01 | 0.002 | 0.1 | 30 | Ag | 5 | 3 | 8 | 30 | 1.0 |
| EXAMPLE 2 | 33 | Sil | 40 | Ag | 0.5 | 0.09 | 1.0 | 30 | Ag | 10 | 4 | 15 | 30 | 1.0 |
| EXAMPLE 3 | 31 | Sil | 40 | Ag | 0.5 | 0.09 | 1.0 | 30 | Ag | 20 | 7 | 35 | 30 | 1.0 |
| EXAMPLE 4 | 28 | Sil | 70 | Ag | 0.5 | 0.09 | 1.0 | 15 | Ag | 10 | 4 | 15 | 15 | 1.0 |
| EXAMPLE 5 | 30 | Sil | 20 | Ag | 0.5 | 0.09 | 1.0 | 40 | Ag | 10 | 4 | 15 | 40 | 1.0 |
| EXAMPLE 6 | 31 | Sil | 20 | Ag | 0.5 | 0.09 | 1.0 | 15 | Ag | 10 | 4 | 15 | 65 | 0.2 |
| EXAMPLE 7 | 31 | Sil | 20 | Ag | 0.5 | 0.09 | 1.0 | 65 | Ag | 10 | 4 | 15 | 15 | 4.3 |
| EXAMPLE 8 | 33 | Sil | 40 | Ag | 0.5 | 0.09 | 1.0 | 30 | Ag | 10 | 4 | 15 | 30 | 1.0 |
| EXAMPLE 9 | 33 | Sil | 40 | Ag | 0.5 | 0.09 | 1.0 | 30 | Ag | 10 | 4 | 15 | 30 | 1.0 |
| EXAMPLE 10 | 30 | Sil | 40 | Ag | 0.5 | 0.09 | 1.0 | 30 | Ag | 10 | 4 | 15 | 30 | 1.0 |
| EXAMPLE 11 | 26 | F | 40 | Ag | 0.5 | 0.09 | 1.0 | 30 | Ag | 10 | 4 | 15 | 30 | 1.0 |
| EXAMPLE 12 | 26 | PEEK | 40 | Ag | 0.5 | 0.09 | 1.0 | 30 | Ag | 10 | 4 | 15 | 30 | 1.0 |
| EXAMPLE 13 | 30 | SiO₂ | 40 | Ag | 0.5 | 0.09 | 1.0 | 30 | Ag | 10 | 4 | 15 | 30 | 1.0 |
| EXAMPLE 14 | 30 | Sil | 40 | Ag | 0.5 | 0.09 | 1.0 | 30 | Ag | 10 | 4 | 15 | 30 | 1.0 |
| EXAMPLE 15 | 33 | Sil | 40 | Au | 0.4 | 0.06 | 0.9 | 30 | Au | 16 | 10 | 25 | 30 | 1.0 |
| EXAMPLE 16 | 31 | Sil | 40 | Cu | 0.1 | 0.03 | 0.5 | 30 | Cu | 13 | 6 | 19 | 30 | 1.0 |
| EXAMPLE 17 | 31 | Sil | 40 | Cu | 0.1 | 0.03 | 0.5 | 30 | Au | 16 | 10 | 25 | 30 | 1.0 |
| COM- | 30 | Sil | 100 | — | — | — | — | — | — | — | — | — | — | — |
| PARISON | | | | | | | | | | | | | | |
| EXAMPLE 1 | | | | | | | | | | | | | | |
| COM- | — | Sil | 20 | Ag | 0.5 | 0.09 | 1.0 | 80 | — | — | — | — | — | — |
| PARISON | | | | | | | | | | | | | | |
| EXAMPLE 2 | | | | | | | | | | | | | | |
| COM- | 30 | Sil | 20 | — | — | — | — | — | Ag | 10 | 4 | 15 | 80 | — |
| PARISON | | | | | | | | | | | | | | |
| EXAMPLE 3 | | | | | | | | | | | | | | |
| COM- | 31 | Sil | 40 | Ag | 0.5 | 0.09 | 1.0 | 30 | Ag | 20 | 7 | 35 | 30 | 1.0 |
| PARISON | | | | | | | | | | | | | | |
| EXAMPLE 4 | | | | | | | | | | | | | | |

### Example 1

The Example 1 is an example in accordance with the electrode portion 1 according to the above-described embodiment.

As shown in Table 1, the stainless steel SUS304 is used as the material of the electrode main body 1A as the base material. The shape of the electrode main body 1A is the round rod shape having a diameter of 0.4 millimetres.

The intermediate layer 1B (reference sign in Table 1 is omitted, and each member name in Table 2 is also omitted) is formed by using the argentum with a layer thickness of 7 micrometres (described as “Ag” in Table 1, and same description is used in other table).

The layer thickness of the intermediate layer 1B is actually measured after the evaluations described below. More specifically, an observation sample is formed by cutting out a cross section of the electrode portion 1 using an ion milling. The layer thickness of the intermediate layer 1B is measured by observing this observation sample using a scanning electron microscope. A measurement method of the layer thickness of the coating layer 1C described below is performed in the same manner.

As shown in Table 2, the layer thickness of the coating layer 1C is 32 micrometres.

The silicone resin (described as “Sil” in Table 2) is used as the material of the base material 5.

The argentum particles are used to form the first metal-particle group. The values of D50, D5, and D95 of the first metal-particle group are 0.01 micrometres, 0.002 micrometres, and 0.1 micrometres respectively. The values of D50, D5, and D95 are three representative values of the particle diameter distribution.

Hereinafter, in order to simplify the description, a group of the values of D50, D5, and D95 will be described as the “representative value” and shown in a format of [D50, D5, D95] with a unit of micrometre.

The measurements of the values of D50, D5, and D95 in the case in which the particle diameter is equal to or less than 1 micrometre are performed by using a dynamic light scattering particle size distribution device. The measurements of the values of D50, D5, and D95 in the case in which the particle diameter exceeds 1 micrometre are performed by using a laser diffraction/scattering particle size distribution device.

The argentum particles are used to form the second metal-particle group. The representative value of the second metal-particle group is [5, 3, 8].

The volume content ratios of the base material 5, the first metal-particle group, and the second metal-particle group in the coating layer 1C (described as “content ratio” is Table 2) are 40 vol %, 30 vol %, and 30 vol % respectively. Accordingly, the volume ratio is 1.0 (=A/B). Here, the reference sign “A” represents the volume content ratio of the first metal-particle group and the reference sign “B” represents the volume content ratio of the second metal-particle group.

The electrode portion 1 is manufactured by the method shown below.

After the electrode main body 1A is manufactured, the argentum is plated on the surface of the electrode main body 1A to form the intermediate layer 1B.

The silicone paint, the first metal-particle group, and the second metal-particle group as the materials of the base material 5 are weighted and then mixed in order to achieve the above-described compounding ratio when cured. Accordingly, the paint for forming the coating layer 1C is manufactured.

The paint is painted on the intermediate layer 1B by the spray painting. Then, the coating film is dried at 200 degrees Celsius for 1 hour. Thus, the electrode portion 1 according to the Example 1 is manufactured.

After connecting the wirings to the electrode portion 1, the grasping portion 2 is attached thereto. The wirings of the electrode portion 1 is electrically connected with the high-frequency power source 3 that is connected by the indifferent plate 4. Thus, the high-frequency knife 10 of the Example 1 is manufactured.

### Examples 2, 3

Examples 2, 3 are different from the Example 1 in each representative value of the first metal-particle group and the second metal-particle group.

The representative values of the first metal-particle group and the second metal-particle group in the Example 2 are [0.05, 0.09, 1.0] and [10, 4, 15] respectively.

The representative value of the first metal-particle group in the Example 3 is same as that in the Example 2. The representative value of the second metal-particle group in the Example 3 is [20, 7, 35].

The layer thickness of the coating layer 1C is 33 micrometres in the Example 2 and 31 micrometres in the Example 3.

The electrode portion 1 and the high-frequency knife 10 according to the Example 2, 3 are manufactured in the same manner with the Example 1 (The same applies to the examples shown below).

### Examples 4-7

The Examples 4-7 are different from the Example 2 in the volume content ratio of each composition.

The volume content ratio of each composition in the Example 4 is 70 vol %, 15 vol %, and 15 vol % in the sequence of the base material 5, the first metal-particle group, and the second metal-particle group. The volume content ratio of each composition in the Example 5 is 20 vol %, 40 vol %, and 40 vol %. Similarly, the volume content ratio of each composition in the Example 6 is 20 vol %, 15 vol %, and 65 vol %. Similarly, the volume content ratio of each composition in the Example 7 is 20 vol %, 65 vol %, and 15 vol %.

Each of the volume ratio A/B in the Example 4 and 5 is 1.0. The volume ratios A/B in the Example 6 and 7 are 0.2 and 4.3 respectively.

The layer thickness of the coating layer 1C in each of the Examples 4-7 is 28 micrometres, 30 micrometres, 31 micrometres, and 31 micrometres.

### Examples 8-10

The Examples 8-10 are different from the Example 2 in the layer thickness of the intermediate layer 1B.

The layer thickness of the intermediate layer 1B in each of the Examples 8-10 is 5 micrometres, 30 micrometres, and 100 micrometres.

The layer thickness of the coating layer 1C in each of the Examples 8-10 is 33 micrometres, 33 micrometres, and 30 micrometres.

### Examples 11-13

The Examples 11-13 are different from the Example 2 in the material for forming the base material 5.

The fluorine resin (described as “F” in Table 2) is used as the base material 5 in the Example 11. The polyetheretherketone resin (described as “PEEK” in Table 2) is used as the base material 5 in the Example 12. The silica as the Ceramics (described as “SiO₂” in Table 2) is used as the base material 5 in the Example 13.

The paint for forming each coating layer 1C is manufactured by mixing the first metal-particle group and the second metal-particle group into each of the fluorine paint, the polyetheretherketone resin, and the silica paint.

The layer thickness of the coating layer 1C in each of the Examples 11-13 is 26 micrometres, 26 micrometres, and 30 micrometres.

### Example 14

The Example 14 is different from the Example 2 in the material of the intermediate layer 1B.

The aluminum (described as “Al” in Table 2) is used as the material of the intermediate layer 1B in the Example 14.

The layer thickness of the coating layer 1C in the Example 14 is 30 micrometres.

### Examples 15-17

The Examples 15-17 are different from the Example 2 in the material and the particle diameter distribution of the first metal-particle group and the second metal-particle group. With regard to the Examples 15-16, they are also different from the Example 2 in the material of the intermediate layer 1B.

The aurum (described as “Au” in Tables 1 and 2) with a layer thickness of 7 micrometres is used as the intermediate layer 1B in the Example 15. The aurum particles with a representative value [0.4, 0.06, 0.9] are used as the first metal-particle group in the Example 15. The aurum particles with a representative value [16, 10, 25] are used as the second metal-particle group in the Example 15.

The cuprum (described as “Cu” in Tables 1 and 2) with a layer thickness of 7 micrometres is used as the intermediate layer 1B in the Example 16. The cuprum particles with a representative value [0.1, 0.03, 0.5] are used as the first metal-particle group in the Example 16. The cuprum particles with a representative value [13, 6, 19] are used as the second metal-particle group in the Example 16.

The cuprum particles with a representative value [0.1, 0.03, 0.5] are used as the first metal-particle group in the Example 17. The cuprum particles with a representative value [16, 10, 25] are used as the second metal-particle group in the Example 17.

The layer thickness of the coating layer 1C in each of the Examples 15-17 is 33 micrometres, 31 micrometres, and 31 micrometres.

### Comparison Examples 1-4

The Comparison Examples 1-4 will be described in focus on the difference from the above-described Examples.

In the Comparison Example 1, the coating layer is formed from the silicone resin only which is same with the Example 1, and the Comparison Example 1 is different from the Example 1 in that the metal particles are not included in the coating layer. The layer thickness of the coating layer in the Comparison Example 1 is 30 micrometres.

In the Comparison Example 2, a coating layer is intentionally manufactured to have the silicone resin with the volume content ratio of 20 vol % that is same with the Example 1 and the argentum particles with the volume content ratio of 80 vol % that is same with the first metal-particle group in the Example 2. However, the viscosity of the paint for forming such a coating layer is too high such that the thin film cannot be formed on the intermediate layer 1B. Accordingly, in Table 2, the layer thickness is described as “-”. With regard to the Comparison Example 2, the evaluations described below cannot be performed.

The Comparison Example 3 is different from the Example 2 in that the coating layer is formed to have the silicone resin with the volume content ratio of 20 vol % that is same with the Example 1 and the argentum particles with the volume content ratio of 80 vol % that is same with the second metal-particle group in the Example 2. The layer thickness of the coating layer in the Comparison Example 3 is 30 micrometres.

The Comparison Example 4 is different from the Example 3 in that the intermediate layer is not formed. The layer thickness of the coating layer in the Comparison Example 4 is 31 micrometres.

(Evaluation Method)

The adhesion preventing characteristic of the electrode portion according to the Examples 1-17 and the Comparison Example 1, 3, 4 with respect to the biological tissues are evaluated.

The evaluation of the adhesion preventing characteristic is performed by measuring the temporal changes of the incision performance of the electrode portion. It is because that when the adhesion of the biological tissues to the electrode occurs, it is difficult to apply the electricity and the incision performance of the electrode degrades.

The specific experiment method is to repeat the incision operation described below.

The stomach of the pig is used as the treatment target. The incision operation of incising the mucosal layer and the submucosal layer of the treatment target is repeated by using the electrode portion of each example and comparison example. One time of the incision operation is performed under a condition of setting the electrode portion in the incision mode with an output of 50 W and determining the incision distance to 10 millimetres.

This incision operation is performed by 500 times per each electrode portion. At the time of the 500th incision, the time (incision time) necessary for forming the 10 millimetres incision is measured.

(Evaluation Result)

In Table 3 shown below, the measured incision time and judgement of the adhesion preventing characteristic evaluation are disclosed therein.

TABLE 3

| | EVALUATION RESULT | |
|---|---|---|
| | INCISION TIME | EVALUATION |
| | (SECOND) | RESULT |
| EXAMPLE 1 | 4 | ○ |
| EXAMPLE 2 | 4 | ○ |
| EXAMPLE 3 | 3 | ○ |
| EXAMPLE 4 | 3 | ○ |
| EXAMPLE 5 | 4 | ○ |
| EXAMPLE 6 | 3 | ○ |
| EXAMPLE 7 | 4 | ○ |
| EXAMPLE 8 | 4 | ○ |
| EXAMPLE 9 | 3 | ○ |
| EXAMPLE 10 | 3 | ○ |
| EXAMPLE 11 | 4 | ○ |
| EXAMPLE 12 | 4 | ○ |
| EXAMPLE 13 | 3 | ○ |
| EXAMPLE 14 | 4 | ○ |
| EXAMPLE 15 | 3 | ○ |
| EXAMPLE 16 | 4 | ○ |
| EXAMPLE 17 | 4 | ○ |
| COMPARISON | 30 | x |
| EXAMPLE 1 | | |
| COMPARISON | — | x |
| EXAMPLE 2 | | |
| COMPARISON | 10 | x |
| EXAMPLE 3 | | |
| COMPARISON | 12 | x |
| EXAMPLE 4 | | |

In a case in which the incision time is equal to or shorter than 5 seconds, the incision performance is favorable. Thus, the adhesion preventing characteristic is determined to be “good” (described as “O” in Table 3).

In a case in which the incision time exceeds 5 seconds, the incision performance is unfavorable. Thus, the adhesion preventing characteristic is determined to be “no good” (described as “X” in Table 3).

As shown in Table 3, the incision time for the electrode portion 1 according to the Examples 1-17 is 3 seconds to 4 seconds. Accordingly, the adhesion preventing characteristic of the electrode portion 1 according to the Examples 1-17 is determined to be “good”.

On the other hand, the incision time for the electrode portion according to the Comparison Examples 1, 3, 4 are 30 seconds, 10 seconds, and 12 seconds respectively. Thus, the adhesion preventing characteristic of the electrode portion according to the Comparison Examples 1, 3, 4 is determined to be “no good”.

With regard to the Comparison Example 2, as described above, the coating layer cannot be formed such that evaluation of the incision time cannot be performed, thus, the electrode portion according to the Comparison Example 2 is determined to be “no good”.

In the case of the Comparison Example 1, the metal particles are not included in the coating layer. Thus, it is difficult for the base material to dissipate the heat to the intermediate layer, and compared to each Example, it is considered that the heat dissipation performance is bad. As a result, it is considerable that the denaturation of the base material due to the heat during the incision operation and the adhesion of the biological tissues are advanced.

In the case of the Comparison Example 3, since the intermediate layer 1B and the second metal-particle group same with the Example 2 are formed, it is considerable that the heat dissipation to the intermediate layer 1B is advanced to a certain degree. However, the contact positions among the metal particles having a relative large diameter are less than the contact positions among the metal particles disclosed in the Examples and the heat dissipation paths are not enough when compared to the electrode disclosed in the Examples such that the denaturation of the base material is advanced.

In the case of the Comparison Example 4, since the first metal-particle group and the second metal-particle group which are same with that in the Example 2 are included, the heat dissipation paths are formed in the coating layer 1C; however, each of the metal particles comes in contact with the electrode main body 1A with a low thermal conductivity such that the heat dissipation is not enough. In other words, in the Comparison Examples, the intermediate layer 1B having the favorable heat dissipation performance as that in each Example is not included, thus the heat dissipation is not enough.

The embodiments of the invention have been described above with reference to the drawings, but specific structures of the invention are not limited to the embodiments and may include various modifications without departing from the scope of the invention. The invention is not limited to the above-mentioned embodiments and is limited only by the accompanying claims.

What is claimed is:

1. An electrode for high-frequency medical device, comprising:
a substrate;
an intermediate layer laminated on the substrate, wherein the intermediate layer has a top layer formed on the outermost part of the intermediate layer and the top layer is a metal layer having a higher thermal conductivity than that of the substrate; and
a coating layer laminated on the intermediate layer, wherein the coating layer is configured such that a plurality of metal particles having a thermal conductivity equal to or larger than 250 W/(m·K) are distributed in a nonmetal material.

2. The electrode for high-frequency medical device according to claim 1, wherein the plurality of metal particles are categorized into at least two groups including a first metal-particle group having a first median diameter and a second metal-particle group having a second median diameter larger than the first median diameter.

3. The electrode for high-frequency medical device according to claim 2,
wherein the first median diameter is equal to or larger than 0.01 micrometres and equal to or less than 0.5 micrometres, and
wherein the second median diameter is equal to or larger than 5 micrometres and equal to or less than 20 micrometres.

4. The electrode for high-frequency medical device according to claim 2,
wherein according to a volume-based cumulative distribution, a particle diameter corresponding to 5% of an accumulation from a small-diameter side toward a large-diameter side is represented as D5 and the particle diameter corresponding to 95% of the accumulation from the small-diameter side toward the large-diameter side is represented as D95,
wherein a value of D95 is equal to or less than 1.0 micrometre in the first metal-particle group, and
wherein a value of D5 is equal to or larger than 3 micrometres, and a value of D95 is equal to or less than 35 micrometres in the second metal-particle group.

5. The electrode for high-frequency medical device according to claim 2, wherein the first metal-particle group and the second metal-particle group are included in the coating layer by a content percentage equal to or more than 10 vol % and equal to or less than 80 vol %.

6. The electrode for high-frequency medical device according to claim 2, wherein a volume ratio of the first metal-particle group with respect to the second metal-particle group is equal to or more than 0.2 and equal to or less than 4.5.

7. The electrode for high-frequency medical device according to claim 1, wherein the nonmetal material of the coating layer includes at least one of a group consisting of fluororesin, silicone resin, polyetheretherketone resin, and ceramic.

8. The electrode for high-frequency medical device according to claim 1, wherein a thickness of the intermediate layer is equal to or more than 5 micrometres and equal to or less than 100 micrometres.

9. The electrode for high-frequency medical device according to claim 1, wherein the electrode includes at least one of a group consisting of a metal material having aluminum, a metal material having titanium, and stainless steel.

10. A high-frequency medical device, comprising the electrode for high-frequency medical device according to claim 1.
